(19) United States
(12) Patent Application Publication
(10) Pub. No.: US 2017/0127738 A1
(43) Pub. Date: May 11, 2017

(54) NOSE WARMING APPARATUS

(71) Applicant: APPAREL HOLDINGS, LLC, NORTHVILLE, MI (US)

(72) Inventors: ANGELA PLACHTA, NORTHVILLE, MI (US); MICHAEL J. BELANGER, NORTHVILLE, MI (US)

(21) Appl. No.: 14/935,640

(22) Filed: Nov. 9, 2015

(51) Int. Cl. A41D 13/11; A61F 11/14; A41D 27/20; A42B 1/02

(52) U.S. Cl. CPC A41D 13/1161; A42B 1/02; A61F 11/14; A41D 27/20

(57) ABSTRACT

A nose warming apparatus for protecting and warming a nose is disclosed. The apparatus includes a nose cover with an outer shell that has a first end and a second end and a top and a bottom for contouring around a nose. A strap is coupled with the nose cover for temporarily securing the nose cover about the nose. The strap extends from a proximal end fixed to the first end of the outer shell to a distal end and includes a VELCRO® fastener for removably attaching the distal end to the second end of the outer shell. An insulated layer is attached to the outer shell. The bottom of the outer shell has an arcuate shape for contouring around the nose while allowing the nose cover to be open adjacent the nostrils as the nose cover engages the nose and enabling unimpeded air flow into and out the nose.

### TECHNICAL FIELD

The present disclosure relates to a nose warming apparatus for protecting and warming a nose.

### BACKGROUND OF THE DISCLOSURE

While enjoying outdoor activities in cooler temperatures, it is common for a person's nose and cheeks to become cold. This problem can be exacerbated by precipitation and wind. Known solutions include full face masks and neck warmers or gaiters that may be pulled over the nose. However, such nose warming apparatuses generally impede air flow through the nostrils of the nose and/or substantially block the mouth. Consequently, undesirable moisture may, for example, build up on the neck gaiter, face mask, or closed nose cover as a result of the humidity of air exhaled through the nose and/or mouth. There are also many instances when it may be desirable to leave parts of the face uncovered (e.g. while eating or drinking). Additionally, known solutions to keep a person's nose warm may not be conveniently utilized and can be misplaced or lost easily when not in use. Accordingly, there is an increasing need for improved nose warming apparatuses.

### SUMMARY OF INVENTION

This section provides a general summary of the present disclosure and is not intended to be interpreted as a comprehensive disclosure of its full scope or all of its features, aspects, and objectives.

Accordingly, it is an aspect of the present disclosure to provide a nose warming apparatus for protecting and warming a nose that can include a nose cover that has an outer shell with a first end and a second end and a top and a bottom and presenting an outer surface and an inner surface for contouring around a nose. The nose warming apparatus can also include a strap coupled with the nose cover for temporarily securing the nose cover about the nose. The strap may extend from a proximal end fixed to the first end of the outer shell to a distal end. The strap may also include a fastener for removably attaching the distal end to the second end of the outer shell. The nose cover can include an insulated layer attached to the inner surface of the outer shell for contacting and insulating the nose. The bottom of the outer shell can have an arcuate shape and extend continuously from the first end to the second end for contouring around a pair of nostrils of the nose while allowing the nose cover to be open adjacent the nostrils as the nose cover engages the nose and enabling unimpeded air flow into and out of the nostrils of the nose.

It is another aspect of the present disclosure to provide a nose warming apparatus for attachment to a piece of apparel to warm a nose and can include a nose cover that has a first end and a second end and a top and a bottom for contouring around a nose. The nose warming apparatus can also include a strap that has a proximal end coupled with the first end of the nose cover and a distal end coupled with the second end of the nose cover. The strap may further include at least one fastener for temporarily securing the nose cover about the nose. More specifically, the strap may comprise a first connecting portion that extends between the proximal end and a first intermediate end adapted to attach to at least one piece of apparel and a second connecting portion that extends between the distal end and a second intermediate end adapted to attach to the at least one piece of apparel.

It is another aspect of the present disclosure to provide a nose warming apparatus for protecting and warming a nose and can include a nose cover that has a first end and a second end for contouring around a nose. The nose warming apparatus can also include a strap that has a proximal end coupled with the first end of the nose cover and a distal end coupled with the second end of the nose cover. The strap can include at least one fastener for temporarily securing the nose cover about the nose. The nose warming apparatus can also include at least one piece of apparel. In detail, the strap may comprise a first connecting portion extending between the proximal end and a first intermediate end attached to the piece of apparel and a second connecting portion extending between the distal end and a second intermediate end attached to the piece of apparel.

One or more aspects of the nose warming apparatus of the present disclosure provide several advantages including unimpeded air flow through the nostrils of the nose and no blockage of the mouth while the nose warming apparatus is used. As a result, undesirable moisture collection around the nose as a result of the humidity of air exhaled through the nose and/or mouth may be minimized. Additionally, the nose warming apparatus is conveniently utilized and may be less likely to become misplaced or lost when not in use.

These and other aspects and areas of applicability will become apparent from the description provided herein. The description and specific examples in this summary are intended for the purpose of illustration only and are not intended to limit the scope of the present disclosure.

### BRIEF DESCRIPTION OF THE DRAWINGS

The drawings described herein are for illustrative purposes only of selected embodiments and not all implementations, and are not intended to limit the present disclosure to only that shown. With this in mind, various features and aspects of example embodiments of the present disclosure will be readily appreciated, as the same becomes better understood by reference to the following detailed description when considered in connection with the accompanying drawings wherein:

FIG. 1A is front view of a nose warming apparatus according to an aspect of the disclosure;

FIG. 1B is a side view of the nose warming apparatus of FIG. 1A;

FIG. 1C is a front view of the nose warming apparatus of FIG. 1A;

FIG. 1D is a perspective view of the nose warming apparatus of FIG. 1A;

FIG. 1E is a cross-sectional view of the nose warming apparatus taken along line 1E-1E of FIG. 1A and illustrates an outer shell and an insulated layer of a nose cover of the nose warming apparatus;

FIG. 1F is a top view of the nose warming apparatus of FIG. 1A;

FIG. 2A is a side view of a nose warming apparatus according to another aspect of the disclosure;

FIG. 2B is a perspective view of the nose warming apparatus of FIG. 2A;

FIG. 3 is a side view of a nose warming apparatus according to still another aspect of the disclosure; and

FIG. 4 is a side view of a nose warming apparatus according to yet another aspect of the disclosure.

### DETAILED DESCRIPTION OF THE ENABLING EMBODIMENTS

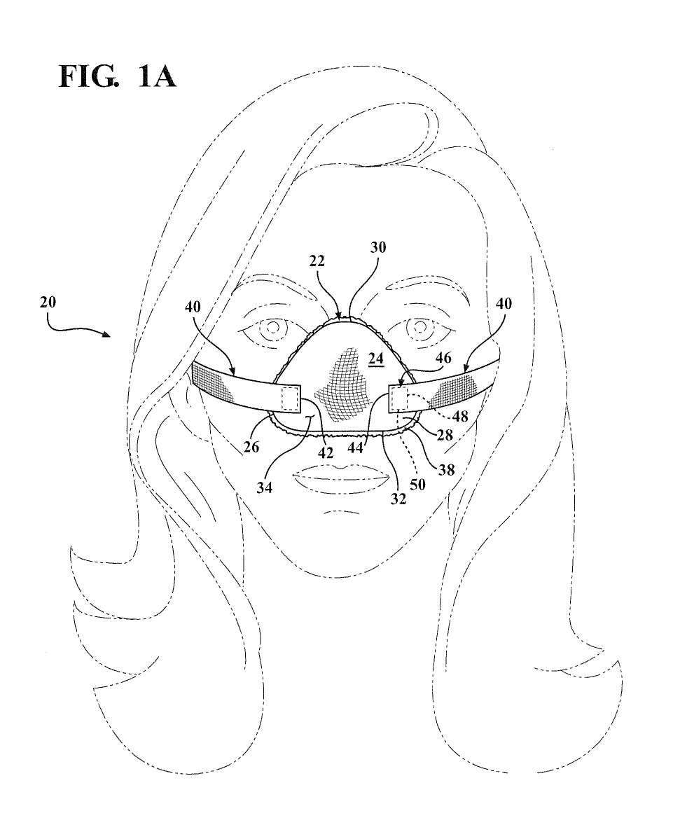

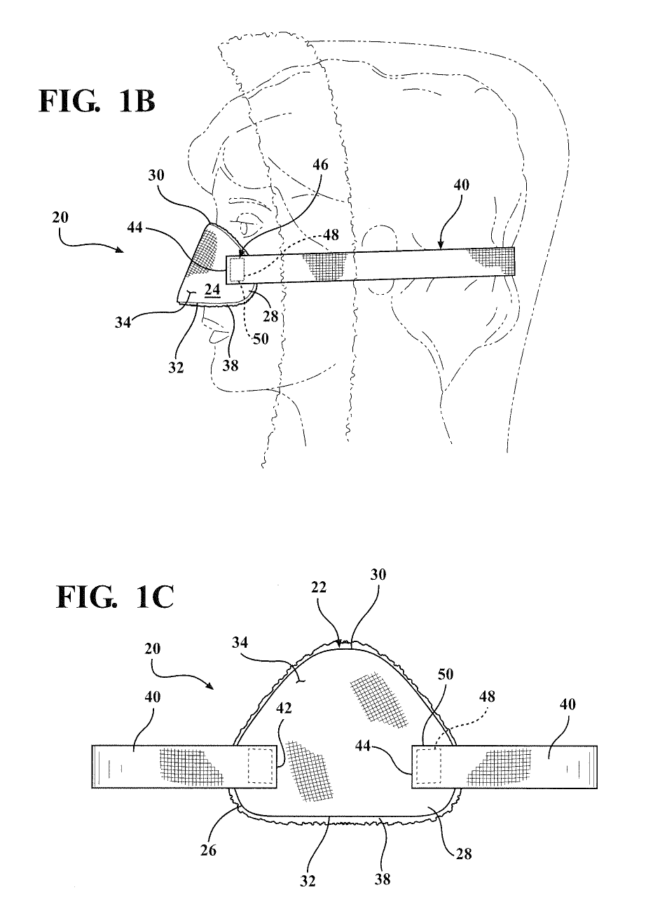

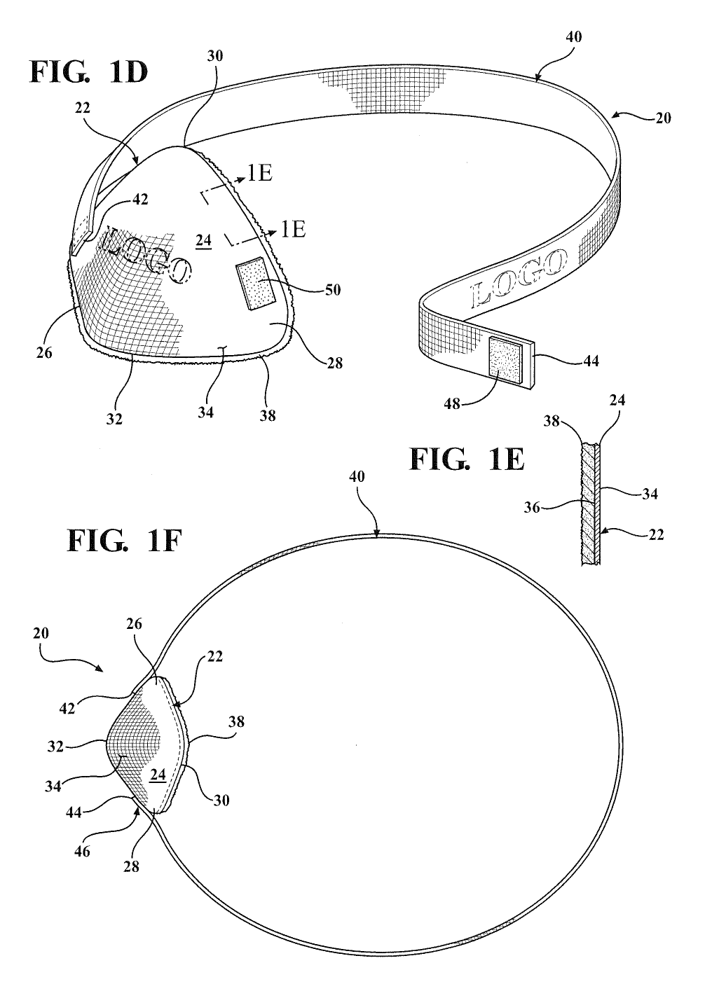

Referring to the Figures, wherein like numerals indicate corresponding parts throughout the several views, a nose warming apparatus 20 for protecting and warming a nose constructed in accordance with aspects of the disclosure is described herein. A nose warming apparatus 20 according to an aspect of the disclosure is shown in FIGS. 1A-1F and can include a nose cover 22 with an outer shell 24 having a first end 26 and a second end 28 and a top 30 and a bottom 32. The outer shell 24 of the nose cover 22 may present an outer surface 34 and an inner surface 36 for contouring around a nose. More specifically, the top 30 of the outer shell 24 can have an arcuate shape that extends continuously from the first end 26 to the second end 28 for engaging a bridge of the nose. According to an aspect of the disclosure, the outer shell 24 can be leather. However, it should be understood that the outer shell 24 could be made of other materials such as, but not limited to an organic polymeric material, Nylon, Gore-tex®, or a metal. A variety of other suitable materials could also be utilized. If the outer shell 24 of the nose cover 22 is metal, for example, the metal may be chosen to be sufficiently flexible to enable the nose cover 22 to contour around the nose.

As best shown in FIG. 1A, the bottom 32 of the outer shell 24 can have an arcuate shape and extend continuously from the first end 26 to the second end 28 for contouring around a pair of nostrils of the nose. Consequently, the nose cover 22 remains open adjacent the nostrils and enables unimpeded air flow into and out of the nostrils of the nose. Because of the unimpeded air flow, outer shell 24 may not be required to be porous and/or breathable meaning that the material of the outer shell 24 can be chosen to prevent moisture and/or wind from penetrating the outer shell 24 from the outer surface 34 to the inner surface 36 (e.g. metal, leather). However, according to other aspects of the disclosure, the material of the outer shell 24 may instead be selected to allow the outer shell 24 to be breathable.

According to an aspect, the nose cover 22 can also include an insulated layer 38 (FIG. 1E) that is coupled with the inner surface 36 of the outer shell 24 for contacting and insulating the nose. The insulated layer 38 can extend from the first end 26 to the second end 28 and from the top 30 to the bottom 32 of the outer shell 24. In other words, the insulated layer 38 can substantially cover the entire inner surface 36 of the outer shell 24. It should be appreciated that the insulated layer 38 may instead cover only a portion of the inner surface 36 of the outer shell 24 (i.e. the insulated layer 38 be comprised of multiple smaller areas). According to another aspect, the insulated layer 38 can be fleece. Yet, it should be understood that the insulated layer 38 could be comprised of other materials in addition to or instead of fleece such as, but not limited to cotton or synthetic foam.

As best shown in FIGS. 1B, 1D and 1F, a strap 40 can be coupled with the nose cover 22 for temporarily securing the nose cover 22 about the nose. The strap 40 may extend between a proximal end 42 fixed to the first end 26 of the outer shell 24 and a distal end 44. The strap 40 can also include a fastener 46 for removably attaching the distal end 44 to the second end 28 of the outer shell 24. That is, according to an aspect, only one end (e.g. the proximal end 42) may be attached to the first end 26 of the nose cover 22 and the other end (e.g. the distal end 44) can be removably attached to the second end 28 of the nose cover 22. However, it should be appreciated that the strap 40 may be coupled with the nose cover 22 in other configurations such as, but not limited to both the proximal end 42 and the distal end 44 being removably attached to the nose cover 22. According to one aspect of the disclosure, the strap 40 may further include at least one elastic or resilient portion and may also include additional loops, and/or buckles (not shown) to allow length adjustment of the strap 40. Alternatively, both ends 42, 44 can be removably attached to the nose cover 22.

According to another aspect of the disclosure, the fastener 46 may include a first patch 48 of VELCRO® attached to the distal end 44 of the strap 40 and a second patch 50 of VELCRO® attached to the second end 28 of the outer shell 24. It should be appreciated that the fastener 46 could also be utilized on the first side of the outer shell 24. The fastener 46 may also comprise various other types of fasteners 46 such as, but not limited to hooks, loops, buttons, snaps, or knots.

In use, the nose warming apparatus 20 according to an aspect of the disclosure may be employed by placing the nose cover 22 over the nose. Next, the strap 40 can be wrapped circumferentially around the back of a user's head. The first patch 48 of VELCRO® located at the distal end 44 of the strap 40 can be placed into engagement with the second patch 50 of VELCRO® on the second end 28 of the outer shell 24 to secure the nose cover 22 about the nose. To remove the nose warming apparatus 20, the first patch 48 located at the distal end 44 of the strap 40 may be disengaged from the second patch 50 on the second end 28 of the outer shell 24.

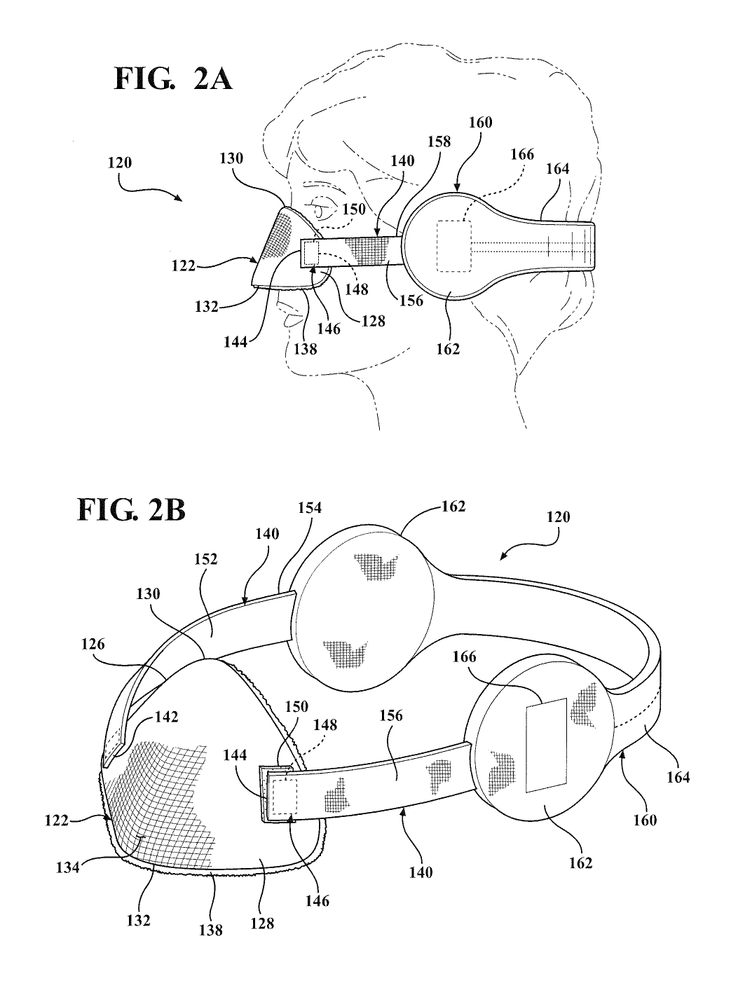

A nose warming apparatus 120 according to another aspect of the disclosure is shown in FIGS. 2A and 2B and includes a nose cover 122 that has a first end 126 and a second end 128 and a top 130 and a bottom 132 for contouring around a nose. The nose warming apparatus 120 also can include at least one piece of apparel. According to an aspect of the disclosure, the piece of apparel can be a pair of ear muffs 160. The ear muffs 160 may include a pair of ear covers 162 disposed in a spaced relationship with one another for engaging ears of a user. The ear muffs 160 may further include a flexible band 164 circuitously extending between and attached to the ear covers 162.

The nose warming apparatus 120 can further include a strap 140 which has a proximal end 142 that is coupled with the first end 126 of the nose cover 122 and a distal end 144 that is coupled with the second end 128 of the nose cover 122. Specifically, the strap 140 may comprise a first connecting portion 152 that extends between the proximal end 142 and a first intermediate end 154 attached to at least one piece of apparel (e.g. the ear muffs 160). Similarly, the strap 140 further may include a second connecting portion 156 extending between the distal end 144 and a second intermediate end 158 attached to the at least one piece of apparel (e.g. the ear muffs 160). Thus, the first connecting portion 152 and second connecting portion 156 of the strap 140 may each attach to one of the ear covers 162 of the ear muffs 160 such that the nose cover 122 can be releasably attached to the ear muffs 160.

The strap 140 can also include at least one fastener 146 for temporarily securing the nose cover 122 about the nose. According to an aspect of the disclosure, the fastener 146 can include a first patch 148 of VELCRO® attached to the distal end 144 of the strap 140 and a second patch 150 of VELCRO® attached to the second end 128 of the outer shell 124. It should be appreciated that the coupling of the strap 140 with the nose cover 122 may alternatively include any type of removable or temporary attachment and/or may also comprise a direct attachment between the strap 140 and the nose cover 122.

According to another aspect, the ear covers 162 of the ear muffs 160 may also include at least one pocket 166 or pouch that may be used to stow the nose warming apparatus 120 when it is not in use. For example, each ear cover 162 could include a pocket 166, one to contain the first connecting portion 152 and nose cover 122, and the other to contain the second connecting portion 156.

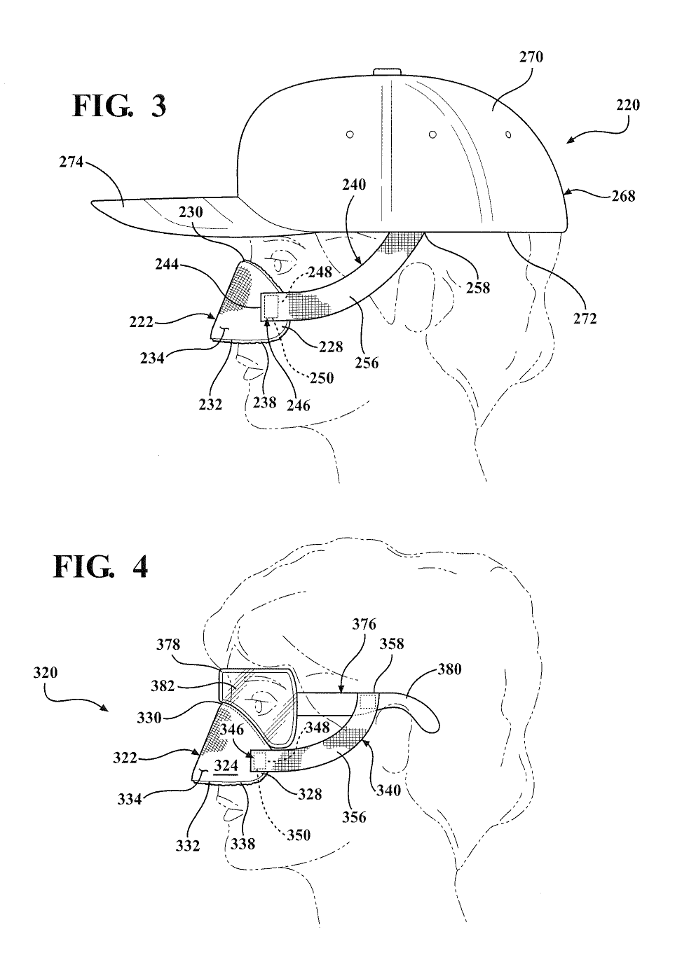

A nose warming apparatus 220 according to another aspect of the disclosure is shown in FIG. 3 and can include a nose cover 222 that has a first end (not shown) and a second end 228 and a top 230 and a bottom 232 for contouring around a nose. The nose warming apparatus 220 also can include at least one piece of apparel. According to an aspect of the disclosure, the piece of apparel can be a hat 268. The hat 268 may include a cup-shaped head cover 270 having a generally circular shaped peripheral band 272 for fitting around a user's head and may also include a brim 274 extending radially outwardly from and attached to the head cover 270.

The nose warming apparatus 220 can also include a strap 240 that has a proximal end (not shown) that is coupled with the first end of the nose cover 222 and a distal end 244 that is coupled with the second end 228 of the nose cover 222. More specifically, the strap 240 may comprise a first connecting portion (not shown) which extends between the proximal end and a first intermediate end adapted attached to the at least one piece of apparel. In the same way, the strap 240 may further include a second connecting portion 256 extending between the distal end 244 and a second intermediate end 258 attached to the at least one piece of apparel. So, the first connecting portion 252 and second connecting portion 256 of the strap 240 may each attach to opposite sides of the peripheral band 272 of the head cover 270.

The strap 240 can also include at least one fastener 246 for temporarily securing the nose cover 222 about the nose. The fastener 246 can include a first patch 248 of VELCRO® attached to the distal end 244 of the strap 240 and a second patch 250 of VELCRO® attached to the second end 228 of the outer shell 224. As with the nose warming apparatus 120 illustrated in FIGS. 2A and 2B, it should be understood that the coupling of the strap 240 with the nose cover 222 may alternatively include any type of removable or temporary attachment and/or may also comprise a direct attachment between the strap 240 and the nose cover 222.

A nose warming apparatus 320 according to another aspect of the disclosure is shown in FIG. 4 and can include a nose cover 322 that has a first end 326 and a second end 328 and a top 330 and a bottom 332 for contouring around a nose. The nose warming apparatus 320 also can include at least one piece of apparel. According to an aspect of the disclosure, the piece of apparel is a pair of sunglasses 376. The sunglasses 376 may include a frame 378 and a pair of temples or arms 380 for fitting around a user's head and also includes a pair of lenses 382. It should be understood that the piece of apparel could instead be a pair of glasses rather than sunglasses 376.

The nose warming apparatus 320 can also include a strap 340 that has a proximal end (not shown) which is coupled with the first end (not shown) of the nose cover 322 and a distal end 344 that is coupled with the second end 328 of the nose cover 322. In detail, the strap 340 may comprise a first connecting portion (not shown) that extends between the proximal end and a first intermediate end attached to at least one piece of apparel. Likewise, the strap 340 may further include a second connecting portion 356 extending between the distal end 344 and a second intermediate end 358 attached to the at least one piece of apparel. Thus, the first connecting portion and second connecting portion 356 of the strap 340 may each attach to one of the arms 380. It should be appreciated that the first connecting portion and second connecting portion 356 of the strap 340 could alternatively attach to other parts of the sunglasses 376, such as, but not limited to the frame 378 adjacent the lenses 382 (e.g. attach to a bridge portion of the frame 378 between the lenses 382).

The strap 340 can also include at least one fastener 346 for temporarily securing the nose cover 322 about the nose. The fastener 346 can include a first patch 348 of VELCRO® attached to the distal end 344 of the strap 340 and a second patch 350 of VELCRO® attached to the second end 328 of the outer shell 324. The coupling of the strap 340 with the nose cover 322 may alternatively include any type of removable or temporary attachment and/or may also comprise a direct attachment between the strap 340 and the nose cover 322.

While the nose warming apparatuses 20, 120, 220, 320 illustrated herein are shown attached to ear muffs 160, a hat 268, and sunglasses 376, for example, it should be appreciated that the nose warming apparatuses 20, 120, 220, 320 may instead be adapted to be removably coupled with the piece of apparel rather than being securely attached. In other words, the nose warming apparatuses 20, 120, 220, 320 could be sold as a kit that may be attached to the piece of apparel. For example, the nose warming apparatus 120 as shown in FIGS. 2A and 2B includes a strap 140 comprising a first connecting portion 152 extending between the proximal end 142 and a first intermediate end 154 adapted to attach to at least one piece of apparel. The second connecting portion 156 could extend between the distal end 144 and a second intermediate end 158 could be adapted to attach to the at least one piece of apparel. The adaptation could comprise features such as, but not limited to the strap 140 including additional reinforcement, patches 148, 150 of VELCRO®, or being looped to fit over a portion of the piece of apparel (e.g. arms 380 of the sunglasses 376). It should also be understood that while nose warming apparatuses 20, 120, 220, 320 illustrated herein are shown attached to ear muffs 160, a hat 268, and sunglasses 376, the piece of apparel could alternatively comprise other garments or accessories such as, but not limited to a hood or collar of a coat or shirt.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure. Those skilled in the art will recognize that concepts disclosed in association with an example switching system can likewise be implemented into many other systems to control one or more operations and/or functions.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known apparatus structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the apparatus in use or operation in addition to the orientation depicted in the figures. For example, if the apparatus in the figures is turned over, elements described as “below” or “beneath” other elements or features would then be oriented “above” the other elements or features. Thus, the example term “below” can encompass both an orientation of above and below. The apparatus may be otherwise oriented (rotated degrees or at other orientations) and the spatially relative descriptions used herein interpreted accordingly.

Obviously, many modifications and variations of the present invention are possible in light of the above teachings and may be practiced otherwise than as specifically described while within the scope of the appended claims. These antecedent recitations should be interpreted to cover any combination in which the inventive novelty exercises its utility. The use of the word “said” in the apparatus claims refers to an antecedent that is a positive recitation meant to be included in the coverage of the claims whereas the word “the” precedes a word not meant to be included in the coverage of the claims.

1. A nose warming apparatus for protecting and warming a nose comprising:
a nose cover including an outer shell having a first end and a second end and a top and a bottom and presenting an outer surface and an inner surface for contouring around a nose,
a strap coupled with said nose cover for temporarily securing said nose cover about the nose,
said strap extending from a proximal end fixed to said first end of said outer shell to a distal end and including a fastener for removably attaching said distal end to said second end of said outer shell,
said nose cover including an insulated layer attached to said inner surface of said outer shell for contacting and insulating the nose, and
said bottom of said outer shell having an arcuate shape and extending continuously from said first end to said second end for contouring around a pair of nostrils of the nose while allowing said nose cover to be open adjacent the nostrils as said nose cover engages the nose and enabling unimpeded air flow into and out of the nostrils of the nose.

2. A nose warming apparatus as set forth in claim 1 further including a piece of apparel and wherein said strap comprises a first connecting portion extending between said proximal end and a first intermediate end coupled to said piece of apparel and a second connecting portion extending between said distal end and a second intermediate end coupled to said piece of apparel.

3. A nose warming apparatus as set forth in claim 2 wherein said piece of apparel is a hat.

4. A nose warming apparatus as set forth in claim 2 wherein said piece of apparel is a pair of ear muffs.

5. A nose warming apparatus as set forth in claim 2 wherein said piece of apparel is a pair of sunglasses.

6. A nose warming apparatus as set forth in claim 1 wherein said insulated layer extends from said first end to said second end and from said top to said bottom of said outer shell.

7. A nose warming apparatus as set forth in claim 1 wherein said outer shell is leather.

8. A nose warming apparatus as set forth in claim 1 wherein said outer shell is metal.

9. A nose warming apparatus as set forth in claim 1 wherein said fastener includes a first patch of VELCRO attached to said distal end of said strap and a second patch of VELCRO attached to said second end of said outer shell for engaging said first patch of VELCRO and securing said nose cover about the nose.

10. A nose warming apparatus for attachment to a piece of apparel to warm a nose comprising:
a nose cover having a first end and a second end and a top and a bottom for contouring around a nose,
a strap having a proximal end coupled with said first end of said nose cover and a distal end coupled with said second end of said nose cover and including at least one fastener for temporarily securing said nose cover about the nose, and
said strap comprising a first connecting portion extending between said proximal end and a first intermediate end adapted to attach to at least one piece of apparel and a second connecting portion extending between said distal end and a second intermediate end adapted to attach to the at least one piece of apparel.

11. A nose warming apparatus as set forth in claim 10 wherein said bottom of said nose cover has an arcuate shape and extends continuously from said first end to said second end for contouring around a pair of nostrils of the nose while allowing said nose cover to be open adjacent the nostrils as said nose cover engages the nose and enabling unimpeded air flow into and out of the nostrils of the nose.

12. A nose warming apparatus as set forth in claim 10 wherein said nose cover includes an outer shell presenting an outer surface and an inner surface and said nose cover including an insulated layer attached to said inner surface of said outer shell for contacting and insulating the nose.

13. A nose warming apparatus as set forth in claim 12 wherein said outer shell is leather.

14. A nose warming apparatus as set forth in claim 12 wherein said outer shell is metal.

15. A nose warming apparatus for protecting and warming a nose comprising:
a nose cover having a first end and a second end for contouring around a nose,
a strap having a proximal end coupled with said first end of said nose cover and a distal end coupled with said second end of said nose cover and said strap including at least one fastener for temporarily securing said nose cover about the nose,
at least one piece of apparel, and
said strap comprising a first connecting portion extending between said proximal end and a first intermediate end attached to said piece of apparel and a second connecting portion extending between said distal end and a second intermediate end attached to said piece of apparel.

16. A nose warming apparatus as set forth in claim 15 wherein said distal end of said strap is removably coupled with said second end of said nose cover and said at least one fastener is disposed at said distal end of said strap.

17. A nose warming apparatus as set forth in claim 15 wherein said piece of apparel is a hat.

18. A nose warming apparatus as set forth in claim 15 wherein said piece of apparel is a pair of ear muffs.

19. A nose warming apparatus as set forth in claim 15 wherein said piece of apparel is a pair of sunglasses.

20. A nose warming apparatus as set forth in claim 15 wherein said piece of apparel further includes at least one pocket for stowing said nose warming apparatus when not in use.
